(19) United States
(12) Patent Application Publication
(10) Pub. No.: US 2022/0000189 A1
(43) Pub. Date: Jan. 6, 2022

(54) WAIST TRIMMER WITH CUT-OUT

(71) Applicant: Patrell Devoux Andrew, Jacksonville, FL (US)

(72) Inventor: Patrell Devoux Andrew, Jacksonville, FL (US)

(21) Appl. No.: 16/919,098

(22) Filed: Jul. 1, 2020

(51) Int. Cl. A41C 1/08; A61F 5/02

(52) U.S. Cl. CPC A41C 1/08; A41B 2400/38; A61F 5/028

(57) ABSTRACT

Implementations of a waist trimmer with cut-out are provided. In some implementations, the waist trimmer with cut-out comprises an inner surface and an opposite outer surface extending lengthwise between a first side and a second side and widthwise between an upper side and a lower side. In some implementations, the waist trimmer with cut-out comprises a curved cutout portion extending from the lower side into the surfaces and a reinforcing attachment tab.

In some implementations, a method of using the waist trimmer with cut-out comprises positioning the cutout portion at the back of the wearer's midsection, wrapping the waist trimmer with cut-out from the first side and the second side around the rest of the midsection including over the abdomen, and securing the waist trimmer with cut-out together around the midsection by the attachment portions of the waist trimmer with cut-out including the reinforcing attachment tab.

### TECHNICAL FIELD

This disclosure relates to implementations of a waist trimmer with cut-out.

### BACKGROUND

A waist trimmer (or waist trimmer belt) is a belt or similar article that is worn around the abdomen and adjacent midsection for slimming efforts and back support. Existing waist trimmers usually cover a limited portion of the abdomen and other intended areas. For example, existing waist trimmers tend to roll up along the upper and lower sides while worn, and this problem worsens the wider the belt is for such coverage.

“Boning”, such as metal or plastic inserts, is often used in existing waist trimmers to try to reduce such rolling. However, the boning is less than fully effective and causes undesirable effects, such as wearer discomfort and reduced durability, as well as increased manufacturing complexity.

### BRIEF DESCRIPTION OF THE DRAWINGS

FIGS. 1 and 2 illustrate an implementation of an example waist trimmer with cut-out according to the present disclosure.

FIGS. 3 and 4 illustrate another implementation of an example waist trimmer with cut-out according to the present disclosure.

FIGS. 5 and 6 illustrate an example use of the waist trimmer with cut-out according to the present disclosure.

### DETAILED DESCRIPTION

Implementations of a waist trimmer with cut-out are provided. In some implementations, the waist trimmer with cut-out comprises an inner surface and an opposite outer surface, a first side and an opposite second side, and an upper side and an opposite lower side. In some implementations, the waist trimmer with cut-out comprises a cutout portion and a reinforcing attachment tab.

In some implementations, the waist trimmer with cut-out is configured to cover a substantial area of a wearer's abdomen when worn.

In some implementations, the waist trimmer with cut-out is configured to not roll/curl up along the upper/lower sides when worn.

In some implementations, the waist trimmer with cut-out is configured to not roll/curl up when worn without the use of boning.

In some implementations, the waist trimmer with cut-out is configured to provide a slimming effect to a wearer's abdomen and/or adjacent midsection when worn.

In some implementations, the waist trimmer with cut-out is configured to provide back support to a wearer when worn.

In some implementations, a method for using the waist trimmer with cut-out comprises positioning the cutout portion at the back of the wearer's midsection. In some implementations, the method comprises wrapping the waist trimmer with cut-out from the first side and the second side around the rest of the midsection including over the abdomen. In some implementations, the method comprises securing the waist trimmer with cut-out together around the midsection by the attachment portions of the waist trimmer with cut-out including the reinforcing attachment tab.

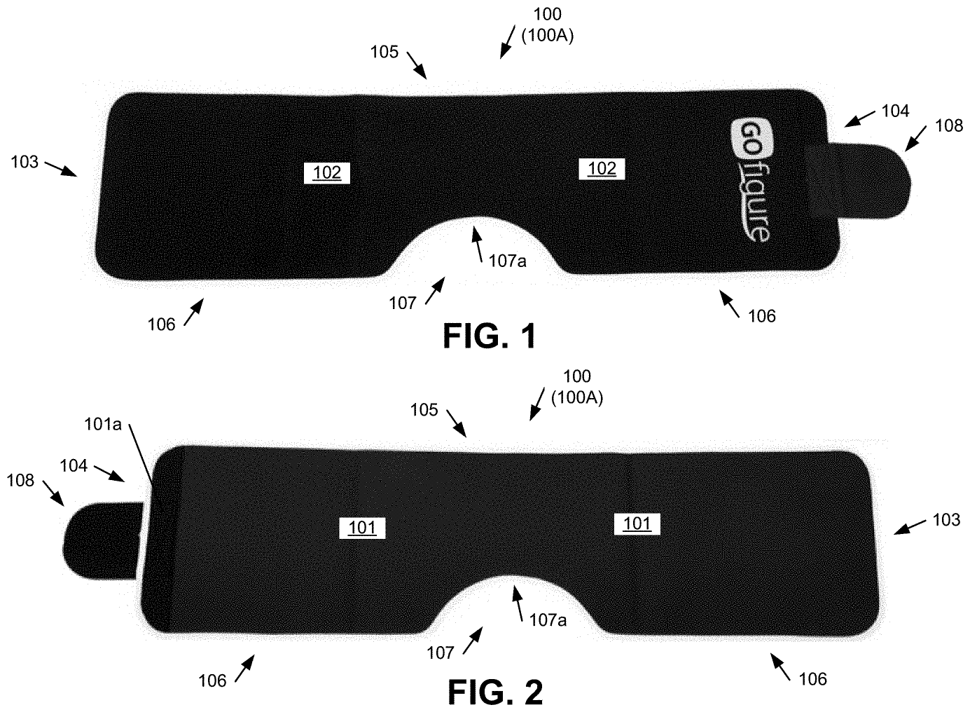

FIGS. 1-2 and 3-4 illustrate implementations of an example waist trimmer with cut-out 100 (100A, 100B) according to the present disclosure. As shown in FIGS. 1 and 2, in some implementations, the waist trimmer with cut-out 100 comprises a first (or inner) surface 101 and a second (or outer) surface 102.

In some implementations, the waist trimmer with cut-out 100 comprises a first side (or edge) 103, a second side (or edge) 104, a third (or upper) side (or edge) 105, and a fourth (or lower) side (or edge) 106. In some implementations, the waist trimmer with cut-out 100 comprises a cut-out (or “cutout”) 107 and a reinforcing attachment portion (or “attachment tab”) 108.

As shown in FIG. 2, in some implementations, the inner surface 101 comprises an attachment portion 101a, as described below.

As shown in FIG. 2, in some implementations, the inner surface 101 extends lengthwise and widthwise. As shown in FIG. 1, in some implementations, the outer surface 102 extends lengthwise and widthwise opposite the inner surface 101.

As shown in FIGS. 1 and 2, in some implementations, the surfaces 101, 102 extend lengthwise between the first side 103 and the second side 104. In some implementations, the surfaces 101, 102 are at least generally elongated between the first side 103 and the second side 104.

In some implementations, the surfaces 101, 102 extend widthwise between the upper side 105 and the lower side 106.

As shown in FIGS. 1 and 2, in some implementations, the surfaces 101, 102 are at least generally rectangular shaped. For example, in some implementations, the surfaces 101, 102 extend lengthwise and widthwise respectively between the sides 103, 104, 105, 106 in an at least generally rectangular shape except for the cutout 107 described below.

In some implementations, the surfaces 101, 102 may be any other suitable shape.

In some implementations, the attachment portion 101a is positioned on at least a portion of the inner surface 101. For example, as shown in FIG. 2, in some implementations, the attachment portion 101a is positioned on the inner surface 101 adjacent to the second side 104.

In some implementations, the attachment portion 101a may extend at least partly between the upper side 105 and the lower side 106. In some implementations, the attachment portion 101a may extend at least partly from or adjacent to the second side 104 toward the first side 103.

In some implementations, the attachment portion 101a may be any suitable shape. For example, as shown in FIG. 2, in some implementations, the attachment portion 101a may be at least generally rectangular shaped.

In some implementations, the attachment portion 101a may be at least generally rectangular shaped such that the attachment portion 101a extends lengthwise at least partly between the upper side 105 and the lower side 106. In some implementations, the attachment portion 101a may be at least generally rectangular shaped such that the attachment portion 101a extends widthwise at least partly from or adjacent to the second side 104 toward the first side 103.

In some implementations, the attachment portion 101a extends widthwise (e.g., at least or approximately) 7 centimeters (or 2.75 inches) from or adjacent to the second side 104 toward the first side 103. In some implementations, the attachment portion 101a may extend widthwise less than 7 centimeters (or 2.75 inches) from or adjacent to the second side 104 toward the first side 103. In some implementations, the attachment portion 101a may extend widthwise more than 7 centimeters (or 2.75 inches) from or adjacent to the second side 104 toward the first side 103.

In some implementations, the attachment portion 101a is configured to removably attach (or connect) to at least a portion of the outer surface 102. In some implementations, the attachment portion 101a is so configured such that at least a portion of the inner surface 101 at/adjacent to the second side 104 can removably attach to at least a portion of the outer surface 102.

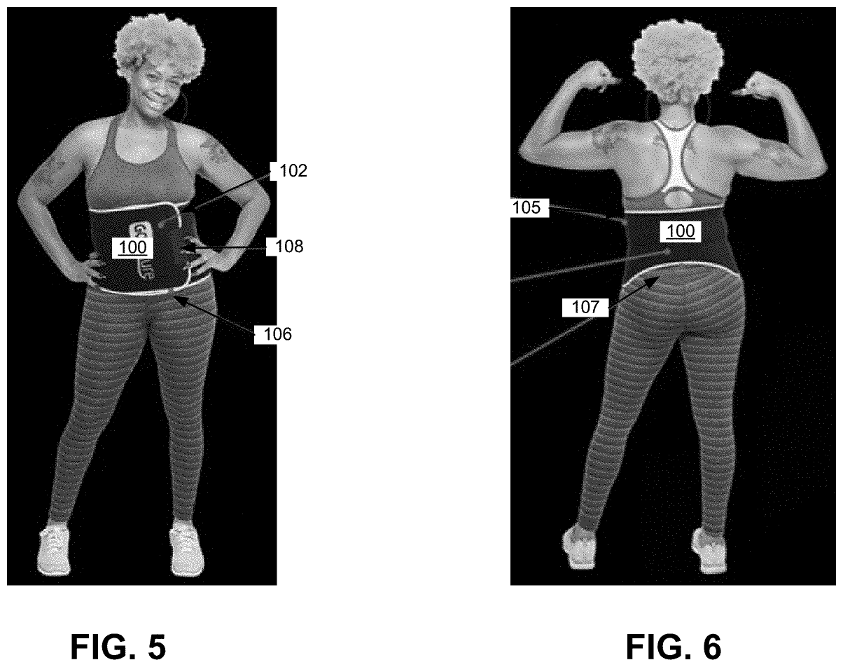

As shown in FIGS. 5 and 6 (described below), in some implementations, the attachment portion 101a is so configured such that the waist trimmer with cut-out 100 can be worn around the midsection of a wearer, such as an area of the body/torso (including the abdomen) between the neck, shoulders, or chest and the legs, hips, or waist. In some implementations, the attachment portion 101a may be so configured such that the waist trimmer with cut-out 100 can be worn in any other suitable way/position on a wearer.

For example, in some implementations, the attachment portion 101a comprises or is composed of a hook material portion of a hook and loop fastener/closure material (e.g., Velcro®). In some implementations, the attachment portion 101a may comprise or be composed of a loop material portion of a hook and loop fastener.

In some implementations, the attachment portion 101a may comprise or be composed of any other suitable material that can removably attach to at least a portion of the outer surface 102. In some implementations, the attachment portion 101a may comprise any other component that can removably attach to at least a portion of the outer surface 102.

In some implementations, the attachment portion 101a is configured to removably attach to at least a portion of the outer surface 102, such as described above, along with the attachment tab 108 (described below) removably attaching to at least a (e.g. adjacent) portion of the outer surface 102. For example, in some implementations, the attachment of the attachment portion 101a to the outer surface 102 is reinforced by the attachment of the attachment tab 108 to the outer surface 102, as described below.

In some implementations, the inner surface 101 is composed of a rubbery material. For example, in some implementations, the inner surface 101 is composed of neoprene or other synthetic rubber material.

In some implementations, the inner surface 101 may be composed of any other suitable material.

In some implementations, the inner surface 101 comprises a grid (e.g., gridded or grid shaped) surface. For example, in some implementations, the inner surface 101 may comprise a plurality of grid (e.g., rectangular, round, etc.) shaped contours/features, such as recesses or projections.

In some implementations, the inner surface 101 may comprise any other suitable surface.

In some implementations, the inner surface 101 is at least (or approximately) 3 millimeters (or 0.125 inches) thick. In some implementations, the inner surface 101 may be less than 3 millimeters (or 0.125 inches) thick. In some implementations, the inner surface 101 may be more than 3 millimeters (or 0.125 inches) thick.

In some implementations, the inner surface 101 may be any other suitable thickness.

In some implementations, the outer surface 102 may be frizz-resistant. For example, in some implementations, the outer surface 102 may be composed of a material or be otherwise configured such that the outer surface 102 is frizz-resistant, such as resistant to curling up, tufting, forming lent, or similar deformation.

In some implementations, the outer surface 102 may be composed of a nylon/spandex blend material. In some implementations, the outer surface 102 may be composed of any other suitable material.

In some implementations, at least a portion of the outer surface 102 may comprise or be composed of a material that is configured to removably attach (or connect) to the attachment portion 101a of the inner surface 101 and/or to the attachment tab 108 (described below).

For example, in some implementations, at least a portion of the outer surface 102 may comprise or be composed of a loop material portion of a hook and loop fastener/closure material (e.g., Velcro®) or of a material that comprises a similar fastening feature/quality. In some implementations, at least a portion of the outer surface 102 may comprise or be composed of a hook material portion of a hook and loop fastener or of a material that comprises a similar fastening feature/quality.

In some implementations, at least a portion of the outer surface 102 may comprise or be composed of any other suitable material that can removably attach to the attachment portion 101a of the inner surface 101 and/or to the attachment tab 108. In some implementations, the outer surface 102 may comprise any other suitable component that can removably attach to the attachment portion 101a and/or to the attachment tab 108.

In some implementations, such portion of the outer surface 102 may extend at least partly from/adjacent to the first side 103 toward/to the cutout 107. In some implementations, such portion of the outer surface 102 may extend at least partly from/adjacent to the first side 103 toward/to any other part of the waist trimmer with cut-out 100.

In some implementations, such portion of the outer surface 102 may also extend at least partly between the upper side 105 and the lower side 106.

In some implementations, such portion of the outer surface 102 may be any suitable shape. For example, in some implementations, such portion of the outer surface 102 may be at least generally rectangular shaped.

As shown in FIG. 1, in some implementations, the first side 103 extends widthwise between the upper side 105 and the lower side 106. In some implementations, the second side 104 extends widthwise between the upper side 105 and the lower side 106 opposite the first side 103.

In some implementations, the sides 103, 104 may be any suitable shape. For example, as shown in FIG. 1, in some implementations, the sides 103, 104 are at least generally straight.

As shown in FIG. 1, in some implementations, the upper side 105 extends lengthwise between the first side 103 and the second side 104. In some implementations, the lower side 106 extends lengthwise between the first side 103 and the second side 104 opposite the upper side 105.

In some implementations, the upper side 105 is at least generally straight. In some implementations, the lower side 106 is at least generally straight except for the cutout 107 described below.

In some implementations, the sides 105, 106 may be any other suitable shape.

In some implementations, the transition/corner between respective sides 103, 104, 105, 106 of the waist trimmer with cut-out 100 may be any suitable shape. For example, as shown in FIG. 1, in some implementations, the transition/corner between the respective sides 103, 104, 105, 106 may be curved.

As shown in FIG. 1, in some implementations, the cutout 107 extends into/through the surfaces 101,102 from the lower side 106. For example, in some implementations, the cutout 107 extends into the rectangular shape of the waist trimmer with cut-out 100 formed by the surfaces 101,102.

In some implementations, the cutout 107 extends from the lower side 106 partly between the first side 103 and the second side 104. For example, in some implementations, the lower side 106 extends straight from the first side 103 to the cutout 107 and from the second side 104 to the cutout 107.

As shown in FIG. 6 (described below), in some implementations, the cutout 107 extends from the lower side 106 such that the cutout 107 positions at least generally at the rear/back side of the midsection of a wearer when wearing the waist trimmer with cut-out 100. In some implementations, the cutout 107 extends from the lower side 106 such that the cutout 107 positions at least generally above/adjacent to the lower back or buttocks of a wearer when wearing the waist trimmer with cut-out 100.

In some implementations, the cutout 107 extends from the lower side 106 such that the cutout 107 positions in any other suitable way.

As shown in FIG. 1, in some implementations, the cutout 107 is curved shaped. For example, in some implementations, the cutout 107 is concaved, such as concave inward into the surfaces 101,102 from the lower side 106.

In some implementations, the cutout 107 is sized and/or shaped such that the top/apex 107a of the cutout is at least (or approximately) 19 centimeters (or 7.5 inches) widthwise from the upper side 105. For example, in some implementations, the cutout 107 curves/concaves inward into the surfaces 101, 102 such that the cutout (e.g., the cutout top 107a) is at least 19 centimeters (or 7.5 inches) widthwise from the upper side 105.

In some implementations, the cutout 107 may be sized and/or shaped such that the top/apex 107a of the cutout is less than 19 centimeters (or 7.5 inches) widthwise from the upper side 105. In some implementations, the cutout 107 may be sized and/or shaped such that the top/apex 107a of the cutout is more than 19 centimeters (or 7.5 inches) widthwise from the upper side 105.

In some implementations, the cutout 107 may be sized and/or shaped such that the top/apex 107a of the cutout is any other suitable distance from the upper side 105.

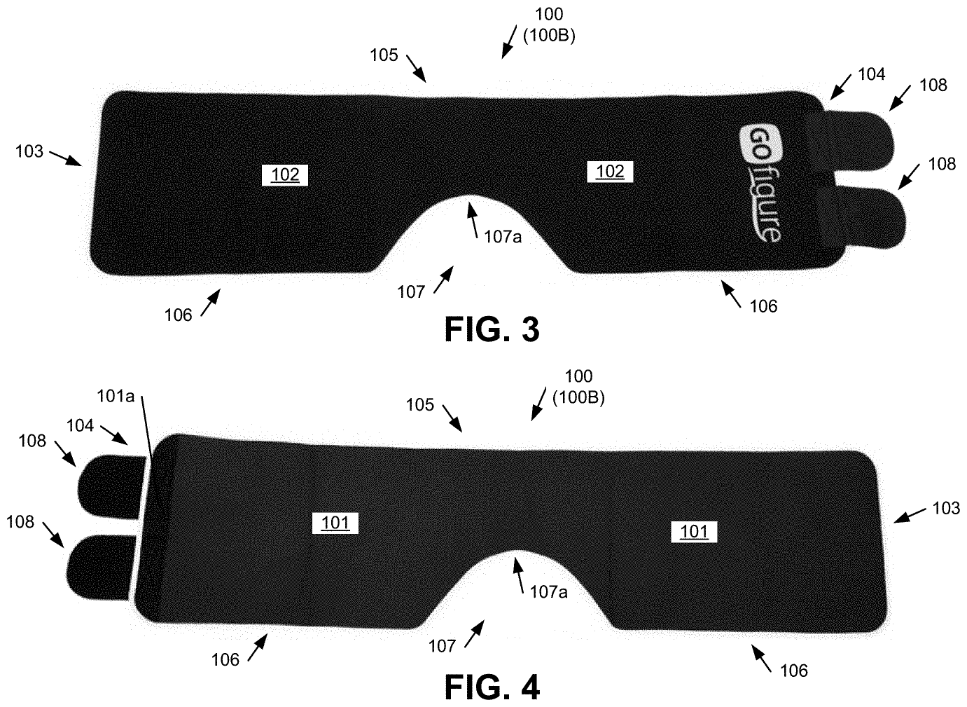

In some implementations, the waist trimmer with cut-out 100 may comprise one or more attachment tabs 108. For example, as shown in FIG. 1, in some implementations, the waist trimmer with cut-out 100 (100A) may comprise one attachment tab 108. As shown in FIG. 3, in some implementations, the waist trimmer with cut-out 100 (100B) may comprise two attachment tabs 108.

In some implementations, the waist trimmer with cut-out 100 (100B) may comprise more than two attachment tabs 108.

In some implementations, the waist trimmer with cut-out 100 may comprise one or more attachment tabs 108 dependent on the dimensions of the waist trimmer with cut-out 100, such as the width between the upper side 105 and the lower side 106.

As shown in FIG. 1, in some implementations, the attachment tab 108 extends/projects from the second side 104. In some implementations, the attachment tab 108 extends from the outer surface 102 at or adjacent to the second side 104.

In some implementations, the attachment tab 108 extends from the second side 104 and/or the outer surface 102 off of or away from the outer surface 102.

In some implementations, the attachment tab 108 extends from the second side 104 and/or the outer surface 102 at least generally perpendicular to the second side 104.

In some implementations, the attachment tab 108 may extend from the second side 104 and/or the outer surface 102 in any other suitable way.

In some implementations, the attachment tab 108 is attached to the second side 104. In some implementations, the attachment tab 108 is attached to the outer surface 102 at or adjacent to the second side 104.

In some implementations, the attachment tab 108 is attached to the outer surface 102 positioned at least generally opposite the attachment portion 101a on the inner surface 101.

In some implementations, the attachment tab 108 is attached to the second side 104 and/or the outer surface 102 positioned at least generally centered widthwise between the upper side 105 and the lower side 106.

In some implementations, the attachment tab 108 is attached to the second side 104 and/or the outer surface 102 positioned in any other suitable location/way.

In some implementations, the attachment tab 108 may be any suitable shape. For example, as shown in FIG. 1, in some implementations, the attachment tab 108 may be at least generally rectangular shaped. In some implementations, the attachment tab 108 may be at least generally curved or rounded on the end opposite the second side 104, e.g. such that the attachment tab 108 is at least generally tongue shaped.

In some implementations, the attachment tab 108 is configured to removably attach (or connect) to at least a portion of the outer surface 102. In some implementations, the attachment tab 108 is so configured such that at least a portion of the attachment tab 108 can removably attach to at least a portion of the outer surface 102.

As shown in FIGS. 5 and 6 (described below), in some implementations, the attachment tab 108 is so configured such that the waist trimmer with cut-out 100 can be worn around the midsection of a wearer, such as an area of the body/torso (including the abdomen) between the neck, shoulders, or chest and the legs, hips, or waist. In some implementations, the attachment tab 108 may be so configured such that the waist trimmer with cut-out 100 can be worn in any other suitable way/position on a wearer.

For example, in some implementations, the attachment tab 108 comprises or is composed of a hook material portion of a hook and loop fastener/closure material (e.g., Velcro®). In some implementations, the attachment tab 108 may comprise or be composed of a loop material portion of a hook and loop fastener.

In some implementations, the attachment tab 108 may comprise or be composed of any other suitable material that can removably attach to at least a portion of the outer surface 102. In some implementations, the attachment tab 108 may comprise any other component that can removably attach to at least a portion of the outer surface 102.

In some implementations, the attachment tab 108 is configured to be a reinforced attachment/latch. For example, in some implementations, the attachment tab 108 is configured to removably attach to at least a portion of the outer surface 102, such as described above, along with the attachment portion 101a removably attaching to at least a (e.g. adjacent) portion of the outer surface 102.

In this way, in some implementations, the attachment tab 108 reinforces/supplements the attachment of the attachment portion 101a to the outer surface 102 when the waist trimmer with cut-out 100 is worn, such as shown in FIGS. 5 and 6 (described below). For example, in some implementations, the attachment of the attachment tab 108 along with the attachment portion 101a to the outer surface 102 provides a more secure and/or firm attachment of the waist trimmer with cut-out 100 around the midsection of a wearer.

As shown in FIGS. 1 and 2, in some implementations, the waist trimmer with cut-out 100 is at least generally elongated between the first side 103 and the second side 104, such as described above about the surfaces 101, 102.

In some implementations, the waist trimmer with cut-out 100 is at least generally rectangular shaped except for the cutout 107 extending from the lower side 106, such as described above about the cutout 107. For example, in some implementations, the waist trimmer with cut-out 100 extends lengthwise and widthwise respectively between the sides 103, 104, 105, 106, such as described above about the surfaces 101, 102.

In some implementations, the waist trimmer with cut-out 100 may be any other suitable shape.

In some implementations, the waist trimmer with cut-out 100 is configured to cover a substantial area of a wearer's abdomen. For example, as shown in FIG. 5 (described below), in some implementations, the waist trimmer with cut-out 100 is sized and/or shaped to position on/over all or most of a wearer's abdomen, abdominals, midriff, etc. when the waist trimmer with cut-out 100 is worn by a user/wearer.

In some implementations, the waist trimmer with cut-out 100 is configured to not roll/curl up (or similarly deform) while worn by a user/wearer. For example, as shown in FIGS. 5 and 6, in some implementations, the waist trimmer with cut-out 100 is configured to cover a substantial area of a wearer's abdomen when worn without the waist trimmer with cut-out 100 rolling up along the upper side 105 and/or lower side 106

In some implementations, the waist trimmer with cut-out 100 is configured to not roll/curl up when worn without the use of boning, such as the metal or plastic inserts, in the waist trimmer with cut-out 100. For example, in some implementations, the waist trimmer with cut-out 100 is configured to not roll up when worn without boning by the thickness of the inner surface 101 and/or outer surface 102, such as the 3 millimeters (or 0.125 inches) thick inner surface 101 described above.

In some implementations, the waist trimmer with cut-out 100 is configured to provide a slimming effect to a wearer's abdomen and/or adjacent midsection when worn.

In some implementations, the waist trimmer with cut-out 100 is configured to provide back support to a wearer when worn.

In some implementations, the waist trimmer with cut-out 100 may be configured to provide any other suitable features.

In some implementations, the waist trimmer with cut-out 100 may be any suitable length (e.g., extending between the first side 103 and the second side 104) and any suitable width (e.g., extending between the upper side 105 and the lower side 106), such as the following examples. In some implementations, the waist trimmer with cut-out 100 may be any suitable length and/or any suitable width respective to the size of the user/wearer, such as the waist size, height, etc. of the user.

In some implementations, the waist trimmer with cut-out 100 may be (e.g., at least or approximately) 89 centimeters (or 35 inches) in length and 23 centimeters (or 9 inches) in width.

In some implementations, the waist trimmer with cut-out 100 may be 104 centimeters (or 41 inches) in length and 28 centimeters (or 11 inches) in width.

In some implementations, the waist trimmer with cut-out 100 may be 122 centimeters (or 48 inches) in length and 33 centimeters (or 13 inches) in width.

In some implementations, the waist trimmer with cut-out 100 may be 137 centimeters (or 54 inches) in length and 38 centimeters (or 15 inches) in width.

In some implementations, the waist trimmer with cut-out 100 may be any other suitable length and any other suitable width, such as less than or greater than the above examples.

In some implementations, the waist trimmer with cut-out 100 comprises any suitable dimensions, such as the example dimensions described above.

In some implementations, the waist trimmer with cut-out 100 is composed of any suitable materials, such as the example materials described above.

In some implementations, the waist trimmer with cut-out 100 can have any suitable appearance, such as the example appearances shown in the FIGS.

FIGS. 5 and 6 illustrate an example use of the waist trimmer with cut-out according to the present disclosure.

In some implementations, an example method of using the waist trimmer with cut-out 100, with respect to the above-described FIGS., comprises positioning the cutout 107 at/adjacent to the back/rear-side of the midsection of the wearer, such as shown in FIG. 6.

In some implementations, the cutout 107 is positioned such that the inner surface 101 is positioned against/adjacent to the wearer's body.

As shown in FIG. 6, in some implementations, the cutout 107 may be positioned such that the cutout 107 is at least generally above/adjacent to the wearer's lower back or buttocks.

In some implementations, the method comprises further positioning the waist trimmer with cut-out 100 around the wearer's midsection, such as shown in FIGS. 5 and 6.

In some implementations, the waist trimmer with cut-out 100 is further positioned such that the inner surface 101 is positioned against/adjacent to the wearer's body.

In some implementations, the waist trimmer with cut-out 100 is further positioned by wrapping the waist trimmer with cut-out 100 around the wearer's midsection such that the waist trimmer with cut-out 100 covers the wearer's abdomen.

In some implementations, the waist trimmer with cut-out 100 is further positioned by wrapping the waist trimmer with cut-out 100 around the wearer's midsection such that the first side 103 and an adjacent portion of the waist trimmer with cut-out 100 to the first side 103 is positioned over at least a portion of the wearer's abdomen. In some implementations, the second side 104 and an adjacent portion of the waist trimmer with cut-out 100 to the second side 104 is positioned over the first side 103 and at least part the adjacent portion of the waist trimmer with cut-out 100 to the first side 103.

In some implementations, the waist trimmer with cut-out 100 is so wrapped such that a respective adjacent portion of the inner surface 101 to the second side 104 is thereby positioned over/adjacent to at least part of a respective adjacent portion of the outer surface 102 to the first side 103.

In some implementations, the method comprises securing the waist trimmer with cut-out 100 as positioned around the wearer's midsection, such as shown in FIG. 5. In some implementations, the waist trimmer with cut-out 100 is secured by removably attaching the attachment portion 101a of the inner surface 101 adjacent to the second side 104 to a respective/adjacent portion of the outer surface 102 adjacent to the second side 104.

In some implementations, the waist trimmer with cut-out 100 is also secured by removably attaching the attachment tab 108 extending from the outer surface 102 adjacent to the second side 104 to a respective/adjacent portion of the outer surface 102 adjacent to the second side 104. In this way, in some implementations, the attachment tab 108 provides a reinforcing latch/attachment of the attachment portion 101a to the outer surface 102.

In some implementations, the method may further comprise removing the waist trimmer with cut-out 100 by detaching the attachment tab 108 and the attachment portion 101a from the respective attachments to the outer surface 102 and by removing the waist trimmer with cut-out 100 from around the wearer's midsection.

In some implementations, the midsection is an area of the wearer's torso that extends at least partly between the neck, shoulders, or chest and the legs, hips, or waist. In some implementations, the midsection is an area of the wearer's body/torso that includes the abdomen or abdominals (e.g., abdominal muscles).

In some implementations, the midsection may comprise the wearer's midriff. In some implementations, the midsection may comprise any other suitable portion of a wearer's body.

In some implementations, the cutout 107 and the positioning thereof prevents the waist trimmer with cut-out 100 from rolling/curling up from the lower side 106 and/or the upper side 105, such as due to the curvature of the wearer's body such as at the lower back/buttocks area. Also, as described above, in some implementations, the thickness of the inner surface 101 and/or the outer surface 102 prevents the waist trimmer with cut-out 100 from rolling/curling up from the sides 105, 106 while the waist trimmer with cut-out 100 is worn.

The figures, including photographs and drawings, comprised herewith may represent one or more implementations of the waist trimmer with cut-out.

Details shown in the figures, such as dimensions, descriptions, etc., are exemplary, and there may be implementations of other suitable details according to the present disclosure.

Reference throughout this specification to “an embodiment” or “implementation” or words of similar import means that a particular described feature, structure, or characteristic is comprised in at least one embodiment of the present invention. Thus, the phrase “in some implementations” or a phrase of similar import in various places throughout this specification does not necessarily refer to the same embodiment.

Many modifications and other embodiments of the inventions set forth herein will come to mind to one skilled in the art to which these inventions pertain having the benefit of the teachings presented in the foregoing descriptions and the associated drawings.

The described features, structures, or characteristics may be combined in any suitable manner in one or more embodiments. In the above description, numerous specific details are provided for a thorough understanding of embodiments of the invention. One skilled in the relevant art will recognize, however, that embodiments of the invention can be practiced without one or more of the specific details, or with other methods, components, materials, etc. In other instances, well-known structures, materials, or operations may not be shown or described in detail.

While operations may be depicted in the drawings in a particular order, this should not be understood as requiring that such operations be performed in the particular order shown or in sequential order, or that all illustrated operations be performed, to achieve desirable results.

1. An article of manufacture, comprising:
a material configured to form a waist trimmer belt having a cutout portion extending on a wearer's back adjacent to the wearer's buttocks and a reinforcing attachment portion for securing the waist trimmer belt, wherein:
the waist trimmer belt comprises an inner surface, an outer surface, a first side, a second side, an upper side, and a lower side, wherein:
the inner surface is elongated extending lengthwise between the first side and the second side and extends widthwise between the upper side and the lower side, wherein the inner surface adjacent to the second side is configured to removably attach to the outer surface;
the outer surface is elongated, opposite the inner surface, extending lengthwise between the first side and the second side and extends widthwise between the upper side and the lower side, wherein at least a portion the outer surface extending from the first side toward the second side is configured to be removably attached to by the inner surface adjacent to the second side and by the reinforcing attachment portion;
the upper side extends straight between the first side and the second side;
the lower side extends straight, opposite the upper side, between the first side and the second side, except that the lower side curves inward toward the upper side partly between the first side and the second side thereby forming the cutout portion of the waist trimmer belt; and
the material extends outward from the second side, away from the first side, at least partly between the upper side and the lower side thereby forming the reinforcing attachment portion, wherein the reinforcing attachment portion is configured to removably attach to the outer surface; and
the waist trimmer belt when worn by the wearer is configured to cover a substantial area of the wearer's abdomen without the material rolling upward from the lower side or downward from the upper side.

2. The article of manufacture of claim 1, wherein the lower side curves inward toward the upper side to an apex at least 7.5 inches from the upper side.

3. The article of manufacture of claim 1, wherein the lower side curves concave inward toward the upper side.

4. The article of manufacture of claim 1, wherein the material does not comprise boning.

5. The article of manufacture of claim 1, wherein:
the inner surface adjacent to the second side comprises a hook and loop closure material portion; and
at least a portion the outer surface extending from the first side toward the second side is configured to be removably attached to by the hook and loop closure material portion of the inner surface.

6. The article of manufacture of claim 5, wherein the hook and loop closure material portion of the inner surface is a hook material portion of the hook and loop closure material that extends at least partly between the upper side and the lower side and extends up to 2.75 inches from the second side toward the first side.

7. The article of manufacture of claim 1, wherein:
the reinforcing attachment portion comprises a hook and loop closure material portion; and
at least a portion the outer surface extending from the first side toward the second side is configured to be removably attached to by the hook and loop closure material portion of the reinforcing attachment portion.

8. The article of manufacture of claim 7, wherein the hook and loop closure material portion of the reinforcing attachment portion is a hook material portion of the hook and loop closure material.

9. The article of manufacture of claim 1, wherein:
the inner surface is composed of a synthetic rubber material; and
the inner surface is at least 0.125 inches thick.

10. The article of manufacture of claim 9, wherein the inner surface is composed of a neoprene material.

11. The article of manufacture of claim 1, wherein the inner surface is gridded.

12. The article of manufacture of claim 1, wherein the outer surface is composed of a frizz-resistant material.

13. The article of manufacture of claim 1, wherein the outer surface is composed of a nylon/spandex blend material.

14. A method of using the article of manufacture of claim 1, comprising:
positioning the cutout portion of the waist trimmer belt on the wearer's back adjacent to the wearer's buttocks;
wrapping the waist trimmer belt from the first side and the second side around the wearer's body including over the wearer's abdomen; and
securing the waist trimmer belt around the wearer's body by removably attaching the inner surface adjacent to the second side to the outer surface and removably attaching the reinforcing attachment portion to the outer surface.

15. An article of manufacture, comprising:
a material configured to form a waist trimmer belt having a cutout portion extending on a wearer's back adjacent to the wearer's buttocks and a reinforcing attachment portion for securing the waist trimmer belt, wherein:
the waist trimmer belt comprises an inner surface, an outer surface, a first side, a second side, an upper side, and a lower side, wherein:
the inner surface is elongated extending lengthwise between the first side and the second side and extends widthwise between the upper side and the lower side, wherein:
the inner surface adjacent to the second side comprises a hook and loop closure material portion configured to removably attach to the outer surface;
the hook and loop closure material portion extends at least partly between the upper side and the lower side and extends up to 2.75 inches from the second side toward the first side;
the inner surface is composed of a synthetic rubber material; and
the inner surface is at least 0.125 inches thick;
the outer surface is elongated, opposite the inner surface, extending lengthwise between the first side and the second side and extends widthwise between the upper side and the lower side, wherein:
at least a portion the outer surface extending from the first side toward the second side is configured to be removably attached to by the hook and loop closure material portion of the inner surface and by a hook and loop closure material portion of the reinforcing attachment portion;
the outer surface is configured to be frizz-resistant; and
the outer surface is composed of a nylon/spandex blend material;
the upper side extends straight between the first side and the second side;
the lower side extends straight, opposite the upper side, between the first side and the second side, except that the lower side, partly between the first side and the second side, curves concave inward toward the upper side to an apex at least 7.5 inches from the upper side thereby forming the cutout portion of the waist trimmer belt;
the material extends outward from the second side, away from the first side, at least partly between the upper side and the lower side thereby forming the reinforcing attachment portion, wherein the reinforcing attachment portion comprises a hook and loop closure material portion configured to removably attach to the outer surface; and
the material does not comprise boning; and
the waist trimmer belt when worn by the wearer is configured to cover a substantial area of the wearer's abdomen without the material rolling upward from the lower side or downward from the upper side.

16. The article of manufacture of claim 15, wherein:
the hook and loop closure material portion of the inner surface is a hook material portion of the hook and loop closure material; and
the hook and loop closure material portion of the reinforcing attachment portion is a hook material portion of the hook and loop closure material.

17. The article of manufacture of claim 15, wherein the inner surface is composed of a neoprene material.

18. The article of manufacture of claim 15, wherein the inner surface is gridded.

19. A method of using the article of manufacture of claim 15, comprising:
positioning the cutout portion of the waist trimmer belt on the wearer's back adjacent to the wearer's buttocks;
wrapping the waist trimmer belt from the first side and the second side around the wearer's body including over the wearer's abdomen; and
securing the waist trimmer belt around the wearer's body by removably attaching the hook and loop closure material portion of the inner surface to the outer surface and removably attaching the hook and loop closure material portion of the reinforcing attachment portion to the outer surface.
